(19) United States
(12) Patent Application Publication
(10) Pub. No.: US 2007/0232996 A1
(43) Pub. Date: Oct. 4, 2007

(54) Balloon for Use in Angioplasty with an Outer Layer of Nanofibers

(75) Inventor: Erik Andersen, Roskilde (DK)

Correspondence Address: BIRCH STEWART KOLASCH & BIRCH, PO BOX 747, FALLS CHURCH, VA, 22040-0747, US

(73) Assignee: Cube Medical A/S, Roskilde (DK)

(21) Appl. No.: 11/587,693

(22) PCT Filed: Apr. 28, 2005
(86) PCT No.: PCT/DK05/00289

§ 371 Date: Feb. 7, 2007

Related U.S. Application Data
(60) Provisional application No. 60/566,087, filed on Apr. 29, 2004.

(30) Foreign Application Priority Data
Apr. 29, 2004 (DK) PA 2004 00671

(51) Int. Cl. A61M 29/00

(52) U.S. Cl. 604/103.02

(57) ABSTRACT

An expandable balloon for use in angioplasty procedures comprises a balloon having an outer surface layer, the outer surface layer being made from electrospun nanofibers and incorporating at least one pharmaceutically active substance, such as nitric oxide (NO). The outer surface layer may be formed on a separate flexible tubular member or sock, which is slipped over the balloon. A method of treating cell disorders in tubular structures of a living being comprises the steps of placing a coated balloon at a treatment site within the tubular structures, expanding the balloon at the treatment site, and releasing the pharmaceutically active substance at the treatment site. Optionally, a stent may be crimped onto the balloon prior to insertion of the balloon and scent into the tubular structures of the living being.

### TECHNICAL FIELD

The present invention relates to a balloon for use in angioplasty and its method of manufacture. The balloon may e.g. be suitable for insertion into the vascular system of a living being, for example for expanding an intravascular stent.

### BACKGROUND OF THE INVENTION

Angioplasty balloons are often used in various diagnostic procedures and medical treatments. For example, balloons are employed to expand stents for implantation in the lumen of a body duct for the treatment of blood vessels exhibiting stenosis. Stents may contain drugs that after implantation elute to the surrounding tissue as to avoid side effects such as cell proliferation. Expandable stents are often placed on an angioplasty balloon catheter which, once in place, is inflated in order to cause the stent to expand. Alternatively, stents may be made from a material which has a recovery capacity such as a super elastic alloy, such as Nitinol, so that the stents may automatically expand, once in place. Such self expanding stents are often delivered by a telescopic tube arrangement where an outer member is removed e.g. by forced sliding over an inner member to which the stent is fixed prior to expansion.

It is generally desired that medical devices for insertion into the vascular system of a living being meet certain physical requirements. For example, the surfaces of stents should be hydrophilic and have a low surface friction in order to facilitate introduction. The stent surfaces may be coated with a pharmaceutical agent, such as nitric oxide (NO). Such nitric oxide releasing matrixes may also relax or prevent arterial spasm once the medical device is in place. Nitric oxide is further known to inhibit the aggregation of platelets and to reduce smooth muscle proliferation, which is known to reduce restenosis. When delivered directly to a particular site, it has been shown to prevent or reduce inflammation at the site where medical personnel have introduced foreign objects or devices into the patient.

International patent application WO 2004/006976 suggests a single layer of lipophilic bioactive material posited or applied to a balloon base material for a direct application to a vessel wall after the previous introduction of another stent. According to the disclosure of the document, the balloon could be used for an angioplasty procedure without the use of a stent. The layer of bioactive material can be posited on the balloon by dipping, soaking or spraying.

Various nictric oxide (NO) donor compounds, pharmaceutical compositions containing such nitric oxide donor compounds and polymeric compositions capable of releasing nitric oxide have also been proposed in the prior art. For example, European patent No. 1220694 B1 corresponding to U.S. Pat. No. 6,737,447 B1 discloses a medical device comprising at least one nanofiber of a linear poly(etihylenimine) diazeniumdiolate forming a coating layer on the device. This polymer is effective in delivering nitric oxide to tissues surrounding medical device.

### SUMMARY OF THE INVENTION

It is an object of preferred embodiments of the present invention to provide a balloon which allows for improved drug delivery in the lumen of a body duct.

In a first aspect, the invention provides an expandable balloon for use in angioplasty procedures, comprising a balloon having an outer surface layer, the outer surface layer being made from nanofibers, such as spun nanofibers, such as electrospun nanofibers, and incorporating at least one pharmaceutically active substance. In a second aspect, the invention provides a method of producing a balloon for use in angioplasty, the method comprising the step of forming an outer surface layer for the balloon by nanofibers, such as by spinning of nanofibers, such as by electrospinning of nanofibers, the outer surface layer containing at least one pharmaceutically active substance. The body portion and the outer surface layer may, for example, define an expandable coated angioplasty balloon, such as a PTA (percutaneous translumenal angioplasty) balloon, a PTCA (percutaneous translumenal coronar angioplasty) balloon or a PTNA (percutaneous translumenal neurovascular angioplasty catheter). Preferably, the outer surface layer is one which conforms to the shape of the balloon, i.e. expands with the balloon when the balloon is inflated and contracts when the balloon is deflated. The outer surface layer is preferably made from a polymer which will be described in further detail below.

Typically, the diameter of the nanofibers is in the range of 2 to 4000 nanometers, preferably 2 to 3000 nanometers, or less than 2000 or less than 1000 nanometers, such as less than 500 or less than 200 nanometers, less than 100 or less than 50 nanometers, such as less than 20 or less than 10 nanometers. Accordingly a large number of nanofibers is present on the outer surface of the balloon. It will thus be appreciated that the nanofibers on the outer surface of the balloon define a large accumulated area, the area being larger with respect to the weight of the balloon than what is achievable with most other non-nanofiber or non-spun surfaces. Accordingly, the surface constitutes a relatively large reservoir for the pharmaceutically active substance compared to the weight of the coated balloon. Nanofibers may even be manufactured to a diameter of 0.5 nanometer which is close to the size of a single molecule.

It has been found that the production of nanofibers by e.g. spinning in many instances be more easily or accurately controlled than methods relying solely on spraying of polymers toward a core. This may confer the further advantage that medical devices may be made with smaller dimensions, such as smaller diameters than hitherto. The present invention allows for the manufacture of balloons with relatively low diameters which, in comparison to devices with larger diameters, facilitate introduction into the vascular system of a living being and reduce side-effects which may occur as a consequence of the introduction of the balloon. The spinning of nanofibers allows for the manufacture of integrated composite devices, in which two or more materials are interlocked on a molecular scale in small dimensions while maintaining a sufficient mechanical stability. Cross-sectional dimensions as small as the dimension of approximately 2-5 molecules of the spun material may be achieved. The size of the molecules evidently depends from the source material used, the size of a polyurethane molecule being usually in the range of less than 3000 nanometers.

One applicable way of producing nanofibers is to form the fibers by electrospinning. It should be understood that the term electrospinning comprises a process wherein particles are applied onto a base element which is kept at a certain, preferably constant, electric potential, preferably a negative potential. The particles emerge from a source which is at another, preferably positive potential. The positive and negative potentials may e.g. be balanced with respect to the potential of a surrounding environment, i.e. a room in which the process is being performed. The potential of the base element with respect to the potential of the surrounding atmosphere may preferably be between −5 and −30 kV, and the positive potential of the source with respect to the potential of the surrounding atmosphere may preferably be between +5 and +30 kV, so that the potential difference between source and base element is between 10 and 60 kV.

The art of producing nanofibers has developed considerably in recent years. U.S. Pat. No. 6,382,526, which is hereby incorporated by reference, discloses a process and apparatus for the production of nanofibers, which process and apparatus are useful in the method according to the present invention, and U.S. Pat. No. 6,520,425, which is hereby incorporated by reference, discloses a nozzle for forming nanofibers. It should be understood that the processes and apparatuses of the aforementioned US patents may be applicable in the method according to the present invention, but that the scope of protection is not restricted to those processes and apparatuses. The fibers may e.g. be spun onto the balloon, as the balloon is continuously rotated, i.e. to form peripherally and/or longitudinally extending strands of nanofibers in the outer surface layer of the balloon.

The balloon may be produced by the present invention may define a plurality of sections along its length. For example, the sections may have different properties, such as different hardness. Such different properties may be arrived at by employing different fiber-forming materials for different sections and/or by changing production parameters, such as voltage of electrodes in an electrospinning process, distance between high-voltage and low-voltage electrodes, rotational speed of the device (or of a core wire around which the device is manufactured), electrical field intensity, corona discharge initiation voltage or corona discharge current.

The body part of the balloon may for example be made of a polyamide material, such as Nylon-12 or Ticoflex™ or a combination thereof. For example, the balloon body may be made from Nylon-12 provided with a coating a Ticoflex™, onto which the outer surface layer is formed by electrospun nanofibers. Alternatively, Ticoflex™ may be used directly as a polymer used for forming the nanofibers.

It has also been found that balloons produced by preferred embodiments of the method according to the invention have a low surface friction. In embodiments of the invention, a low surface friction may be achieved by applying a hygroscopic material as a fiber forming material for the fiber forming process, e.g. the electrospinning process. Accordingly, once introduced into the vascular system, the hygroscopic material absorbs bodily fluid, resulting in a hydrophilic low-friction surface. A hygroscopic surface may for example be achieved with a polyurethane or a polyacrylic acid material.

Preferably, the outer surface layer of the balloon may constitute a reservoir to drugs. The nanofiber portions thereof constitute reservoirs for holding drugs or constitute a matrix polymer source where the drug is either blocked into the molecule chain or adheres to or surrounds the molecule chain. The balloons disclosed herein may carry any appropriate drug, including but not limited to nitric oxide compositions, heparin and chemotherapeutical agents.

The outer surface layer of the expandable balloon may be made from nanofibres which incorporate at least one pharmaceutically active substance. The fibres may form a polymer matrix of one or more polymers. It should be understood that the “outer surface layer made from fibres”, i.e. the polymer matrix, needs not to be the outermost layer of the balloon, for example a layer of a hydrophilic polymer (e.g. polyacrylic acids (and copolymers), polyethylene oxides, poly(N-vinyl lactams such as polyvinyl pyrrolidone, etc.) may be provided as a coating on the outer surface layer (polymer matrix). Alternatively, a barrier layer may be provided as coating on the outer surface layer (polymer matrix) in order to ensure that contact between the polymer matrix and blood is delayed until the expandable balloon is in place. The barrier layer may either be formed of a biodegradable polymer which dissolves or disintegrates, or the barrier layer may be disintegrate upon inflation of the balloon.

By the term “polymer matrix” is meant the three-dimensional structure formed by the electrospun fibers. The polymer matrix may be characterized by a very high accessible surface area which allows swift liberation of the pharmaceutically active substance(s). The polymer of the polymer matrix may be prepared from various polymer-based materials and composite matrixes thereof, including polymer solutions and polymer melts. Applicable polymers are, e.g., polyamides including nylon, polyurethanes, fluoropolymers, polyolefins, polyimides, polyimines, (meth)acrylic polymers, and polyesters, as well as suitable co-polymers. Further, carbon may be used as a fiber-forming material.

The polymer matrix is formed of one or more polymers and may—in addition to the pharmaceutically active substance(s)—incorporate or comprise other ingredients such as salts, buffer components, microparticles, etc.

By the term “incorporates at least one pharmaceutically active substance” is meant that the pharmaceutically active substance(s) is/are either present as discrete molecules within the polymer matrix or is/are bound to the polymer(s) of the matrix either by covalent bonds or by ionic interactions. In the latter of the two instances, the pharmaceutically active substance(s) typically needs to be liberated from the polymer molecules before the biological effect can enter into effect. Liberation will often take place upon contact with physiological fluids (e.g. blood) by hydrolysis, ion-exchange, etc.

In one preferred embodiment, the pharmaceutically active substance is covalently bound to polymer molecules.

The pharmaceutically active substance may be mixed into a liquid substance from which the outer surface layer is manufactured.

In one interesting embodiment, the pharmaceutically active substance is a nitric oxide donor. For certain medical treatments, it is desired that nitric oxide is released into the body tissue in the gas phase immediately upon placement of the balloon at the treatment site, or within 5 minutes at most from its placement. As nitric oxide is released in the gas phase, it may be achieved that no or only few residues of the NO donor are deposited in the tissue.

In preferred embodiments of the present invention, NONO'ates are applied as nitric oxide donors. NONO'ates break down into the parent amine and NO gas in an acid catalyzed manner, according to the below figure, cf. U.S. Pat. No. 6,147,068, Larry K. Keefer: Methods Enzymol, (1996) 268, 281-293, and Naunyn-Schmeideberg's Arch Pharmacol (1998) 358, 113-122.

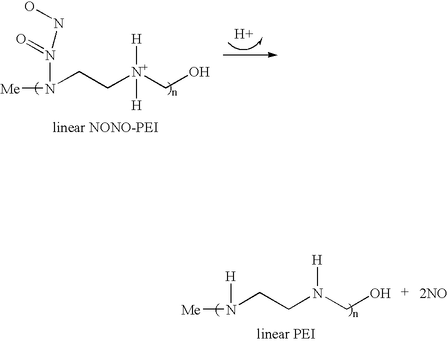

In this embodiment, NO is released within the polymer matrix formed e.g. by spinning, such as electrospinning. As the matrix is porous, water may enter into the matrix. The NO molecule can be transported out of the matrix and into the tissue in a number of ways and combinations hereof. In the following some scenarios are described: NO becomes dissolved in water within the matrix and transported out of the matrix by diffusion or by water flow; NO diffuse out of the matrix in gas form and becomes dissolved in water outside the matrix; NO diffuses from water into the tissue; NO diffuses all the way from the matrix in gas form into the tissue.

As illustrated in the above figure, the rate of NO liberation highly depends on the pH of the media. Thus, by addition of various amounts of an acid to the matrix, the rate of NO liberation can be controlled. As an example, the half-live of NO liberation at pH=5.0 is approximately 20 minutes whereas at pH=7.4 the half-live is approximately 10 hours. As an example, Ascorbic Acid can be used as an acidic agent for enhancing release of NO.

Various nitric oxide (NO) donor compounds and polymeric compositions capable of releasing nitric oxide have also been proposed in the prior art, e.g. U.S. Pat. No. 5,691,423, U.S. Pat. No. 5,962,520, U.S. Pat. No. 5,958,427, U.S. Pat. No. 6,147,068, and U.S. Pat. No. 6,737,447 B1 (corresponding to EP 1220694 B1), all of which are incorporated herein by reference.

In preferred embodiments, the nanofibers are made from polymers which have nitric oxide donors (e.g. a diazeniumdiolate moiety) covalently bound thereto.

Polyimines represent a diverse group of polymer which may have diazeniumdiolate moieties covalently bound thereto. Polyimines include poly(alkylenimines) such as poly(ethylenimines). For example, the polymer may be a linear poly(ethylenimine) diazeniumdiolate (NONO-PEI) as disclosed in U.S. Pat. No. 6,737,447 which is hereby incorporated by reference. The loading of the nitric oxide donor onto the linear poly(ethylenimine) (PEI) can be varied so that 5-80%, e.g. 10-50%, such as 33%, of the amine groups of the PEI carry a diazeniumdiolate moiety. Depending on the applied conditions, the linear NONO-PEI can liberate various fractions of the total amount of releasable nitric oxide.

Polyamines with diazeniumdiolate moieties (in particular poly(ethylenimine) diazenium-diolate) may advantageously be used as a polymer for the nanofiber-forming process by e.g. spinning such as electrospinning because such polymers typically have a suitable hydrophilicity and because the load of diazeniumdiolate moieties (and thereby the load of latent NO molecules) can be varied over a broad range, cf. the above example for NONO-PEI.

In another embodiment, the pharmaceutically active substance(s) is/are present within the polymer matrix as discrete molecules.

Within this embodiment, it the pharmaceutically active substance(s) may be contained in microparticles, such as microspheres and microcapsules. Such microparticles are in particular useful in the treatment of cancer. The microparticles may be biodegradable and may be made from a biodegradable polymer such as a polysaccharide, a polyamino acid, a poly(phosphorester) biodegradable polymer, a polymers or copolymers of glycolic acid and lactic acid, a poly(dioxanone), a poly(trimethylene carbonate)copolymer, or a poly(α-caprolactone) homopolymer or copolymer.

Alternatively, the microparticles may be non-biodegradable, such as amorphous silica, carbon, a ceramic material, a metal, or a non-biodegradable polymer.

The microparticles may be in the form of microspheres that encapsulate the pharmaceutically active substance, such as the chemotherapeutic agent. The release of the pharmaceutically active substance preferably commences after the administration.

The encapsulating microspheres may be rendered leaky for the pharmaceutically active substance by means of an electromagnetic or ultrasound shock wave.

In order to facilitate passage of the balloon to the treatment site along an often tortuous path, a hydrophilic layer is preferably applied to the outer surface layer. The hydrophilic layer may be provided as a separate layer of material. Alternatively, the outer surface layer may itself exhibit hydrophilic properties.

The outer surface layer may advantageously include an acidic agent, such as lactic acid or vitamin C, which acts as a catalyst for releasing the pharmaceutically active substance, e.g. nitric oxide. The acidic agent is capable of changing the ph-value at the treatment site, the release rate of nitric oxide at the treatment site varying as a function of the local ph-value. Thus, the presence of vitamin C may boost the nitric oxide release, i.e. provide a shock-like release of nitric oxide.

In general, the release of nitric oxide is described in Prevention of intimal hyperplasia after angioplasty and/or stent insertion. Or, How to mend a broken heart by Jan Harnek M D, Heart Radiology, University of Lund, Sweden, 2003.

The pharmaceutically active substance may be provided in the form of biodegradable beadings distributed between the nanofibers, the beadings being capable of releasing the pharmaceutically active substance and, in the case of biodegradable beadings, to degrade following release. Such beadings, which are described in more detail in WO 2005/018600 which is hereby incorporated by reference in its entirety, may penetrate into the tissue at the treatment site and release the pharmaceutically active substance there. Alternatively, they may be of a size which is so small that they may be transported away, e.g. with the flow of blood, away from the treatment site.

The outer surface layer may be formed on a separate flexible tube or “sock” which is slipped over the balloon. Accordingly, various flexible tubes having various properties or incorporating various pharmaceutically active substances may be inexpensively manufactured and slipped over traditional, mass manufactured balloons. The flexible tube may be formed by providing a core element, such as a mandrel, onto which the nanofibers are deposited by e.g. spinning, such as electrospinning, as the mandrel is continuously rotated.

In an unexpanded state of the balloon, the flexible tube may be folded around, so that the flexible tube, when seen in cross-section, defines a spoke-and-hub-formation.

In order to improve adhering of the outer layer to the balloon body, the balloon body may be covered by an intermediate polymer layer, such as a Ticoflex™ layer, before it is being coated. For example, the intermediate layer may be formed by dip-coating the balloon body. The intermediate layer may alternatively be formed by a polyurethan or by the polymer which is also used for the outer surface coating, e.g. a linear poly(ethylenimine) diazeniumdiolate as disclosed in U.S. Pat. No. 6,737,447 B1. Dip coating is known per se. For example, dip coating is used in the rubber industry for the manufacture of latex products, and co-extrusion is e.g. applied in the manufacture of fibre-optics cables. Braiding may be employed as an alternative to dip-coating for achieving a roughened or textured surface.

In a further aspect, the invention provides a method of treating cell disorders, such as inflammation, proliferation or cancer, in tubular structures of a living being, comprising the steps of:
placing a balloon as discussed above at a treatment site within the tubular structures;
expanding the balloon at the treatment site;
releasing the pharmaceutically active substance at the treatment site.

The step of releasing the pharmaceutically active substance may be controlled by the presence of a ph-controlling substance incorporated in the outer surface layer, e.g. an acidic agent, such as C vitamin (ascorbic acid) or lactic acid.

Prior to the step of placing the balloon, an unexpanded stent may be placed on the balloon, which stent may be placed at the treatment site along with the balloon. In such an embodiment, the stent is subsequently expanded at the treatment site as the balloon is being expanded, and finally the balloon is deflated and removed from the tubular structure while the stent is left at the treatment site. This confers the advantage that the delivery of the pharmaceutically active substance does not commence fully until inflation of the balloon, and that delivery is substantially interrupted as soon as the balloon is deflated and removed, so that the time of delivery may be accurately controlled. Moreover, the amount of drug which is lost when the stent is conveyed through tubular structures of the living being to the treatment site may be reduced.

In a yet further aspect, the invention also provides a kit comprising a coated balloon as described above, a stent and optionally a guide wire for guiding the stent to the treatment site.

### BRIEF DESCRIPTION OF THE DRAWINGS

Embodiments of the invention will now be further described with reference to the drawing, in which:

FIGS. 1-6 are step-by-step illustrations of a preferred embodiment of a method for producing a medical tubing, e.g. a tubular member for an embodiment of a balloon according to the present invention;

FIG. 7 shows an embodiment of an angioplasty balloon catheter comprising a balloon according to the present invention;

FIGS. 8 and 9 illustrate folding of a balloon.

### DETAILED DESCRIPTION OF THE DRAWING

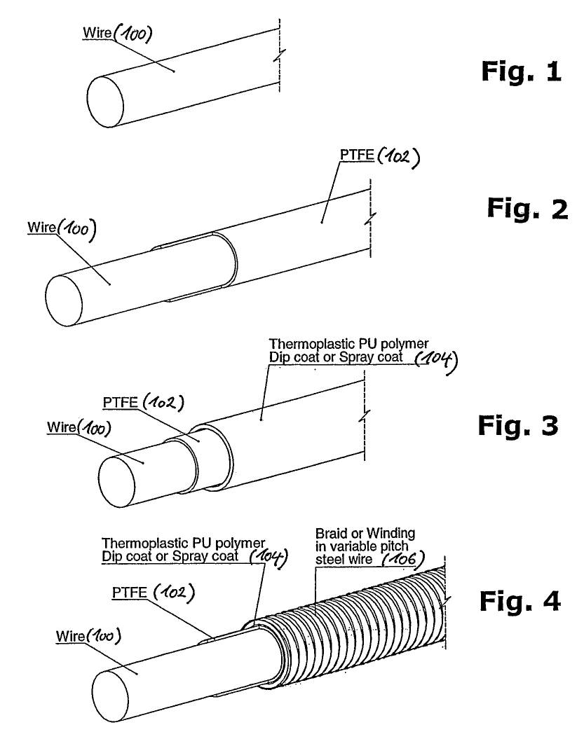

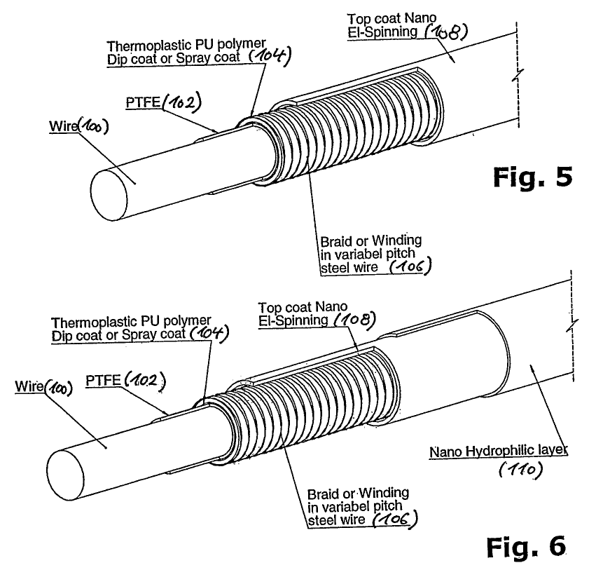

In the embodiment of FIGS. 1-6, the nanofibers are spun onto an outer surface of a core member. The core member comprises a core wire (or mandrel) 100, a layer 102 of PTFE applied to an outer surface of the core wire, a coating 104 of a thermoplastic material applied to an outer surface of the PTFE layer 102, and at least one reinforcing wire 106 applied to an outer surface of the thermoplastic coating, with the filaments of nanofibers being provided as an outer layer 108, i.e. enclosing the reinforcing wire and the thermoplastic coating. The nanofibers may e.g. be produced as devised in U.S. Pat. No. 6,382,526 or U.S. Pat. No. 6,520,425 and subsequently spun onto the intended target object, e.g. during rotation thereof. The nanofibers may likewise be formed by electrospinning, also during continuous rotation of the target object. A hydrophilic layer 110 is optionally applied to an outer surface of the device, cf. FIG. 6.

The diameter of the guide wire may be at least 0.1 mm, such as in the range of 0.1 to 1.0 mm or larger. The thermoplastic coating, which is preferably a coating of polyurethane (PU), preferably has a thickness of 5 μm to about 0.05 mm, preferably 0.01 mm±20%. The reinforcing wire(s) preferably has/have a diameter of 5 μm to about 0.05 mm, preferably 0.01 mm±20%.

As described above, a layer of PTFE 102 may be applied to an outer surface of the core member 100. At least a portion of the surface of the layer of PTFE, such as the portion onto which the nanofibers and/or the thermoplastic coating are to be applied, may be modified for improved bonding of material to the outer surface of the PTFE layer. Preferably, such modifying comprises etching, which may for example result in a primed PTFE surface for covalent bonding or gluing. Etching may be achieved by applying a flux acid or hydroflouric acid to a surface of the PTFE layer. The layer of PTFE may be provided as a hose which is slipped over and co-extends with the core wire.

A coating of a thermoplastic material 104, such as polyurethan (PU), may be provided to an outer surface of the core member 100, i.e. to an outer surface of the PTFE layer 102 in case such a layer has been provided. Following the step of providing the layer of PTFE 102 and/or the step of providing the thermoplastic coating 104, one or more reinforcing wires 106 may be applied to an outer surface of the core member 100, i.e., in a preferred embodiment, to an outer surface of the polyurethane coating 104. The reinforcing wire(s) may consist of one or wires made from steel or/and wires made from yarn, such as carbon filament, which may be applied by winding. Alternatively, the reinforcing wire may be applied by spinning of nanofibers, such as by electrospinning as described above. The reinforcing wire may be formed from carbon or polymer, including polymer solutions and polymer melts. Applicable polymers are: nylon, fluoropolymers, polyolefins, polyimides, and polyesters.

While forming the tubular member, or at least while forming that portion of the tubular member which is formed by nanofibers, e.g. by electrospinning, the core member 100 is preferably rotated, so as to evenly distribute the nanofibers around the outer surface of the core member.

In a preferred embodiment of the invention, nanofibers 108 are applied to the outer surface of the core member at this stage, that is preferably to the outer surface of the thermoplastic coating 104 which is optionally reinforced by the reinforcing wire(s).

A solvent, such as tetrahydroforane (THF) or isopropanol alcohol (IPA), may subsequently be applied to an outer surface of the core member, the outer surface being defined by the nanofiber portion (or layer) 108 of the device. The thermoplastic coating 104 thereby at least partially dissolves in the solvent, so as to bond the reinforcing wire(s) 106 thereto. The reinforcing wire(s) 106 thereby become(s) embedded in the thermoplastic coating 104. It has been found that the step of providing the solvent results in a highly dense surface with a low surface friction, which is believed to be due to crumpling or shrinking of stretched molecules of nanofibers once the solvent is applied.

The core wire 100 (or mandrel) is removed from the device following the step of applying the solvent or prior to the step of applying solvent but subsequent to the step of applying the filament of nanofibers 108.

The resulting tubular member may be used as a flexible tube or sock which may be slipped over a balloon.

Alternatively, nanofibers may be formed directly onto the balloon by electrospinning, the balloon being optionally coated, e.g. dipcoated, or braided as discussed above to enhance adhering of the nanofibers to its surface.

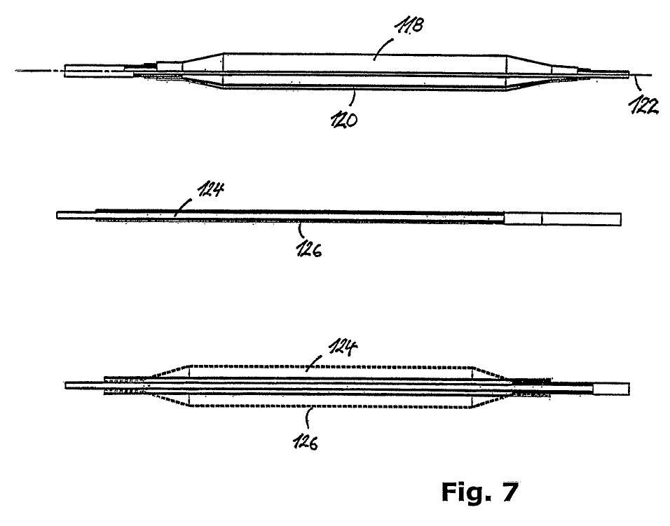

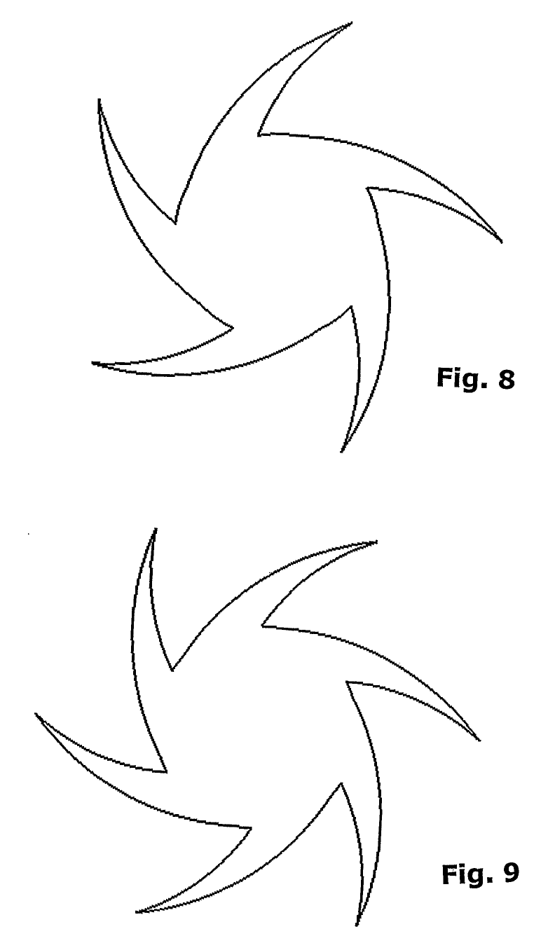

FIG. 7 shows different embodiments of an angioplasty balloon catheter comprising a balloon in accordance with the present invention. In the upper drawing of FIG. 7 there is shown an inflated balloon 118 which comprises an outer surface layer 120 made from electrospun nanofibers. The balloon is carried by a guidewire 122.

The middle drawing of FIG. 7 shows a non-inflated balloon 124 over which there is slipped a tube or “sock” 126 made from electrospun nanofibers. In the lower drawing of FIG. 7, the dashed lines show the contour of the balloon 124 and the sock 126 when the balloon is inflated.

FIGS. 8 and 9 are schematic illustrations of an unexpanded state of a balloon, wherein a flexible tube is folded, so that the flexible tube, when seen in cross-section, defines a spoke-and-hub-formation.

1. An expandable balloon for use in angioplasty procedures, comprising a balloon having an outer surface layer, the outer surface layer being made from nanofibers and incorporating at least one pharmaceutically active substance.

2. A balloon according to claim 1, further comprising an intermediate layer formed between the balloon and the outer surface layer, the intermediate layer being formed by dip-coating.

3. A balloon according to claim 1, wherein the outer surface layer is formed on a separate flexible tube and the outer surface layer is slipped over the balloon.

4. A balloon according to claim 3, wherein the flexible tube is folded, so that the flexible tube, when seen in cross-section, defines a spoke-and-hub-formation.

5. A balloon according to claim 1, wherein the pharmaceutically active substance comprises nitric oxide, and wherein the outer surface layer optionally further includes an acidic agent.

6. A balloon according to claim 1, wherein the outer surface layer is essentially made from a polymer matrix, which contains molecules capable of releasing the at least one pharmaceutically active substance.

7. A balloon according to claim 6, wherein the outer surface layer is essentially made from a polymeric linear poly(ethylenimine) diazeniumdiolate.

8. A balloon according to claim 1, wherein the pharmaceutically active substance is provided in the form of biodegradable beadings distributed between the nanofibers.

9. A balloon according to claim 1, wherein the outer surface layer is formed from spun nanofibers, such as electrospun nanofibers.

10. A kit comprising a stent and a coated balloon according to claim 1 for expanding the stent.

11. A kit according to claim 10, further comprising a guide wire for guiding the stent to a treatment site in tubular structures of a living being.

12. A kit according to claim 11, wherein the guide wire is provided with a coating.

13. A method of producing a balloon for use in angioplasty, the method comprising the step of forming an outer surface layer for the balloon by nanofibers, the outer surface layer containing at least one pharmaceutically active substance.

14. A method according to claim 13, wherein the outer surface layer is formed by spinning, such as by electrospinning.

15. A method according to claim 14, wherein the outer surface layer is applied in the unexpanded state of the balloon.

16. A method according to claim 13, further comprising, prior to the step of forming the outer surface layer, a step of dip-coating the balloon to form an intermediate layer.

17. A method according to claim 13, comprising:
forming the outer surface layer on a separate flexible tube;
slipping the flexible tube over the balloon.

18. A method according to claim 17, wherein the step of forming the outer surface layer on the flexible tube comprises:
providing at least one core member;
forming the flexible tube with the outer surface layer by electrospinning the nanofibers onto an outer surface of the core member.

19. A method according to claim 17 or 18, further comprising, subsequent to the step of slipping the flexible tube over the balloon, folding the flexible tube, so that the flexible tube, when seen in cross-section, defines a spoke-and-hub-formation.

20. A method according to claim 13, wherein the pharmaceutically active substance comprises nitric oxide.

21. A method according to claim 20, wherein the outer surface layer further comprises an acidic agent.

22. A method according to claim 13, wherein the outer surface layer is essentially made from a polymer matrix, which contains molecules capable of releasing the at least one pharmaceutically active substance.

23. A method according to claim 22, wherein the outer surface layer is essentially made from a polymeric linear poly(ethylenimine) diazeniumdiolate.

24. Use of an acidic agent as catalyst for the release of nitric oxide in a balloon according to claim 1.

25. A method of treating cell disorders in tubular structures of a living being, comprising the steps of:
placing a balloon according to claim 1 at a treatment site within the tubular structures;
expanding the balloon at the treatment site.
releasing the pharmaceutically active substance at the treatment site.

26. A method according to claim 25, wherein the step of releasing is controlled by the presence of a ph-controlling substance contained in the outer surface layer.

27. A method according to claim 24, further comprising, prior to the step of placing the balloon, placing an unexpanded stent on the balloon; and placing the stent at the treatment site along with the balloon; and subsequently expanding the stent at the treatment site as the balloon is being expanded; and subsequently deflating the balloon and removing it from the tubular structure while the stent is left at the treatment site.
